(19) United States
(12) Patent Application Publication
(10) Pub. No.: US 2003/0152646 A1
(43) Pub. Date: Aug. 14, 2003

(54) Method of treating conditions caused by activated microglia

(76) Inventors: Valter D. Longo, Los Angeles, CA (US); Caleb E. Finch, Altadena, CA (US); Zhong Xie, Los Angeles, CA (US)

Correspondence Address: HOGAN & HARTSON L.L.P., 500 S. GRAND AVENUE, SUITE 1900, LOS ANGELES, CA, 90071-2611, US

(21) Appl. No.: 10/316,372

(22) Filed: Dec. 11, 2002

Related U.S. Application Data
(60) Provisional application No. 60/341,182, filed on Dec. 12, 2001.

(51) Int. Cl. A61K 33/00

(52) U.S. Cl. 424/718

(57) ABSTRACT

This invention provides methods of preventing neurotoxicity by activated microglia. A method of treating a medical condition in a subject is provided, wherein said condition is affected by the presence of neurotoxins such as peroxynitrite or TNF-α, said method comprising administering to the subject a compound that decomposes peroxynitrite or inhibits TNF-α secretion but does not affect normal activity of microglia, wherein the decomposition of peroxynitrite alone is sufficient to alleviate the pathology of said condition. The method of this invention may be used in conjunction with the administration of a vaccine that increases the microglial activity of clearing Aβ

### FIELD OF THE INVENTION

The present invention relates to the study and treatment of neurodegenerative disorders. In particular, the present invention relates to methods of preventing neurotoxicity by activated microglia, including but not limited to microglia activated by amyloid β-peptide (Aβ)₁₋₄₂, anti-Aβ-antibodies, a combination of Aβ₁₋₄₂ and anti-Aβ-antibodies, or LPS.

### BACKGROUND OF THE INVENTION

Throughout this application, various publications are referenced by author and date. Full citations for these publications may be found listed at the end of the specification immediately preceding the claims. The disclosures of these publications are hereby incorporated by reference in their entireties into this application in order to more fully describe the state of the art as known to those skilled therein as of the date of this invention described and claimed herein.

Microglial activation is implicated in many neurodegenerative disorders, including Alzheimer's disease, multiple sclerosis, Parkinson's disease, and stroke. Alzheimer's disease, the most common form of senile dementia, is accompanied by a progressive loss of neurons and synapses in brain regions characterized by senile plaques and neurofibrillary tangles. The major components of senile plaques are the β-amyloid (Aβ) peptides, which in experimental models can damage neurons directly, or indirectly through the activation of microglia [Yanker, 1990; Pike, 1991; Meda, 1995; Combs, 2001; Klein, 2001].

The amyloid β-peptide (Aβ) promotes the activation of microglia and the generation of cytokines and oxygen species, including nitric oxide (NO) and tumor necrosis factor α (TNF-α), which can be either neurotoxic or neuoprotective. Activated microglia are capable of releasing neurotoxic molecules such as proinflammatory cytokines (e.g., TNF-α), nitric oxide, and superoxide [Colton, 1987; Klegeris, 1994]. Accumulating evidence shows activated microglia can damage or kill neurons in vitro by generating neurotoxic agents including nitric oxide [Chao, 1992; Boje, 1992; Goodwin, 1995; Meda, 1995], tumor necrosis factor-a (TNF-α) [Wood, 1995], various toxic oxygen species [Tanaka, 1994], L-cysteine [Yeh, 2000, phenolic amine [Giulian, 1995], and tissue plasminogen activator [Flavin, 2000]. Nitric oxide (NO) and superoxide (O₂.⁻) react to form the neurotoxic peroxynitrite (ONOO⁻) [Koppal 1999; Estevez, 1998; Estevez, 1998] which has been implicated is Alzheimer's disease, in part because the levels of nitrotyrosine, a product of the reaction of peroxynitrite with tyrosine, increase in Alzheimer's disease [Smith, 1997].

Alzheimer's disease brains show widespread oxidative damage [Mattson, 1997; Smith 2000]. Both hydrogen peroxide and superoxide have been implicated in the direct toxicity of AB to neurons [Behl, 1994; Behl, 1997; Keller, 1998; Longo, 2000], whereas nitric oxide has been implicated in the neurotoxicity of microglia activated by Aβ [Chao, 1992; Boje, 1992; Goodwin, 1995; Meda 1995]. Aβ also stimulates superoxide production in microglia [McDonald, 1997; Klegeris, 1997; Colton, 2000], apparently by the activation of a cell membrane-associated NADPH oxidase [Bianca, 1999]. The activation of HADPH oxidase in Alzheimer's disease [Shimohama, 2000] is consistent with a role of peroxynitrite, the product of the reaction between nitric oxide and superoxide, in Aβ neurotoxicity [Iadecola, 1999].

Although NO can be neurotoxic, NO is also an important signaling molecule that can protect PC12 cells and primary neurons against Aβ toxicity (Troy, 2000; Wirtz-Brugger, 2000). Furthermore, the protective effect of inhibitors of NO synthase (NOS) against Aβ toxicity [Ii, 1996] may be attributable to the inhibition of neuronal instead of microglial inducible nitric oxide synthase (iNOS), as suggested by studies of monocytes stimulated with Aβ [Combs, 2001]. Although NO has been implicated in Aβ₁₋₄₂ toxicity, the role of NO is controversial since the NO donor SNAP was reported by others to be neuroprotective against Aβ toxicity [Troy, 2000; Wirtz-Brugger, 2000]. These differences may be explained by the absence of microglia in the neuronal cultures exposed to Aβ and SNAβ. Whereas microglia generate both superoxide and nitric oxide, and consequently peroxynitrite, the concentration of superoxide in neurons exposed to Aβ in the absence of activated microglia may be too low to react with the NO generated by SNAP and produce neurotoxic levels of peroxynitrite. The sub-toxic dose (100 μM) of the NO donor SNAP used in these studies may prevent the increase of superoxide generation which occurs in neurons treated with Aβ in the absence of microglia [Keller, 1998; Longo, 2000]. In fact, NO activates guanylate cyclase and increases the generation of the guanosine 3′, 5′-cyclic monophosphate (cGMP), which protects against cell death [Kim 1999; Wirtz-Brugger, 2000].

Smith [2002] discloses a vaccine for treating Alzheimer's disease which increases microglial activation. It is also known that microglia are responsible for the clearance of B-amyloid from the brain of mice in response to vaccination with the β-amyloid peptide. In a human trial [Smith 2002], a percentage of patients vaccinated with β-amyloid developed severe inflammation-dependent side effects which led to the premature termination of the clinical trial. Treatment with anti-inflammatory drugs helped the patients recover, suggesting that microglia are generating toxic molecules while clearing Aβ. As proposed Smith et al., [2002], oxidants may mediate the inflammation-dependent toxicity.

However, a role of peroxynitrite in the toxicity of Aβ or activated microglia has not been demonstrated. Therefore, the mechanisms of Aβ and microglial neurotoxicity remain unclear.

### SUMMARY OF THE INVENTION

One aspect of this invention is based on the recognition that peroxynitrite (ONOO⁻), formed by the reaction of nitric oxide (NO) with superoxide (O₂.⁻), is a major mediator in neurotoxicity. This invention is further based on the discovery of a direct link between Aβ activation of microglia and Alzheimer's disease. More specifically, it was discovered compounds that block or decompose peroxynitrite alone without affecting the normal activity of microglia are sufficient to treat neurogenerative diseases caused by activated microglia.

Accordingly, one aspect of this invention provides a method of treating a medical condition in a subject, wherein said condition is affected by the presence of peroxynitrite, said method comprising administering to the subject a compound that decomposes peroxynitrite but does not negatively affect the normal activity of microglia and other brain cells, wherein the decomposition of peroxynitrite alone is sufficient to alleviate the pathology of said condition. In one embodiment, said medical condition is Alzheimer's disease.

Yet another aspect of this invention provides a method of treating a medical condition in a subject, wherein said condition is affected by Aβ₁₋₄₂-activation of microglia, said method comprising administering to the subject a compound that decomposes peroxynitrite but does not negatively affect the normal activity of microglia, wherein the decomposition of peroxynitrite alone is sufficient to alleviate the pathology of said condition.

Another aspect of this invention is based on a method of treating Alzheimer's disease using peroxynitrite decomposition catalysts that block the toxicity of microglia that are activated by anti-Aβ-antibodies and as a result generate oxidants in an attempt to clear Aβ from the brain. Accordingly, another embodiment of this invention comprises a method of treating a medical condition in a subject, wherein said condition is affected by the presence of peroxynitrite, said method comprising administering to a subject a vaccine that increases the microglial activity of clearing Aβ, wherein the method further comprises administering a compound that decomposes peroxynitrite but does not negatively affect the normal activity of microglia and other brain cells, wherein the decomposition of peroxynitrite alone is sufficient to alleviate the pathology of said condition. The compound that decomposes peroxynitrite may be added before, concurrently, or after administration of the vaccine.

This invention is further based on the discovery that compounds that inhibit TNF-α secretion alone without negatively affecting the normal activity of microglia are sufficient to treat neurogenerative diseases caused by microglia. Accordingly, another aspect of this invention provides a method of treating a medical condition in a subject, wherein said condition is affected by the presence of TNF-α, said method comprising administering to the subject a compound that inhibits secretion of TNF-α from microglia but does not negatively affect the normal activity of said microglia, wherein the inhibition of TNF-α alone is sufficient to alleviate the pathology of said condition.

In accordance with another aspect of the present invention, there is provided a method for identifying mediators of Aβ and LPS neurotoxicity by studying the role of inhibitors of specific molecules released by activated microglia in preventing cell death in neurons.

In accordance with another aspect of the present invention, there is provided a method for identifying mediators of Aβ and LPS neurotoxicity by studying the role of decomposition catalysts of specific molecules released by activated microglia in preventing cell death in neurons.

It is a further aspect of the present invention to identify or develop therapeutic compounds that protect neurons against the toxicity of specific molecules such as peroxynitrite or TNF-α without interfering with the normal functions of microglia, wherein the protection against peroxynitrite or TNF-α alone is sufficient to prevent neurotoxicity.

Neurotoxicity according to the methods of this invention is studied in a co-cultures system in which microglia and neurons can be separated before cell death analysis. Accordingly, this invention further provides a method of screening an effective test compound that decreases neuron death caused by a neurotoxin, said method comprising:

(a) providing a co-culture of microglia and neurons;

(b) exposing said co-culture to said test compound to form a test mixture;

(c) subjecting said test mixture to conditions that activate said microglia;

(d) examining said test mixture at a selected time after said subjection for the extent of neuron cell death; and

(e) measuring the generation of nitric oxide, wherein said effective test compound is identified as a compound that decreases neuron death relative to a control sample and has little or no effect on the generation of nitric oxide.

This invention demonstrates that cell death in co-cultures of microglia-neurons activated by, for example, lipopolysaccharide (LPS) or Aβ₁₋₄₂, follows a peak in the generation of superoxide and nitrite and is caused by short-lived diffusible molecules. In one embodiment, LPS or Aβ₁₋₄₂-induced neurotoxicity is blocked by inhibitors of NO synthesis and by peroxynitrite decomposition catalysts such as FeTMPyP and FTPPS. The TNF-α inhibitor pentoxifylline, which does not reduce NO generation, only slightly decreases the toxicity of activated microglia. The specificity of FeTMPyP for peroxynitrite was confirmed by its ability to block the neurotoxicity of low levels of the NO/superoxide donor SIN-1. Moreover, FeTMPyP did not protect neurons against a donor of NO or yeast mutants lacking superoxide dismutases (SODs). These results demonstrate for the first time that peroxynitrite mediates the toxicity of activated microglia and plays a major role in Aβ₁₋₄₂ neurotoxicity.

The inventors are the first to demonstrate that it is sufficient to decompose peroxynitrite generated by microglia exposed to human Aβ to block neurotoxicity without affecting the normal activity of microglia. Therefore, the data presented herein suggests that peroxynitrite decomposition catalysts can be used to treat Alzheimer's disease without affecting the normal function of microglia and other brain cells. Peroxynitrite decomposition catalysts can also be used to block the toxicity of microglia that are activated by anti-Aβ-antibodies and generate oxidants in an attempt to clear Aβ from the brain.

Additional advantages and features of this invention shall be set forth in part in the description that follows, and in part will become apparent to those skilled in the art upon examination of the following specification or may be learned by the practice of the invention. The features and advantages of the invention may be realized and attained by means of the instrumentalities, combinations, and methods particularly pointed out in the appended claims.

### BRIEF DESCRIPTION OF THE DRAWINGS

The accompanying drawings, which are incorporated in and form a part of the specification, illustrate non-limiting embodiments of the present invention, and together with the description serves to explain the principles of the invention.

In the Figures:

FIG. 1A is a graph of neuron survival and nitrite accumulation versus LPS concentration after a 48-hour treatment of rat microglia-neuron co-cultures with 0.2 to 100 ng/ml LPS. Viable neurons were detected by fluorescein diacetate staining. Data represent mean±SE from a representative of 3 independent experiments.

FIG. 1B is a graph of the time course of nitrite and superoxide production as determined by Griess reaction and EPR analysis, respectively. The experiment was repeated three times with similar results. A representative experiment is shown.

FIG. 1C shows scans of superoxide/peroxynitrite generation by microglia cells. Microglia cells (100,000) with or without LPS treatment (24 hours) were incubated with 120 mm DMPO in the presence or absence of either 2 μM SOD (LPS+SOD) or 200 mM DMSO (LPS+DMSO) for 15 minutes and analyzed by EPR. Scans shown are an accumulation of seven scans.

FIG. 2A is a bar graph of neuron survival in microglia-neuron co-cultures treated with 100 ng/ml LPS in the presence or absence of 1 mM of the NOS inhibitor L-NMMA for 48 hours.

FIG. 2B is a bar graph of nitrite accumulation in microglia-neuron co-cultures treated with 100 ng/ml LPS in the presence or absence of 1 mM of the NOS inhibitor L-NMMA for 48 hours.

FIG. 2C is a bar graph of TNF-α secretion in microglia-neuron co-cultures treated with 100 ng/ml LPS in the presence or absence of 1 mM NOS inhibitor L-NMMA for 48 hours. For FIGS. 2A-2C, nitrite and TNF-α were quantified from the same cultures used to measure neuron survival. Values show mean±SEM from at least 5 independent experiments (a=p<0.05 compared with LPS).

FIG. 3A is a bar graph of neuron survival in microglia-neuron co-cultures treated with 100 ng/ml LPS for 48 hours in the presence or absence of either pentoxifylline (PEN, 500 μM) or thalidomide (THA, 200 μM).

FIG. 3B is a bar graph of nitrite accumulation in microglia-neuron co-cultures treated with 100 ng/ml LPS for 48 hours in the presence or absence of either pentoxifylline (PEN, 500 μM) or thalidomide (THA, 200 μM).

FIG. 3C TNF-α secretion in microglia-neuron co-cultures treated with 100 ng/ml LPS for 48 hours in the presence or absence of either pentoxifylline (PEN, 500 μM) or thalidomide (THA, 200 μM). In FIGS. 3A-3C: values show mean±SEM from 5 to 8 independent experiments (a=p<0.05 compared with LPS).

FIG. 4A is a bar graph of neuron survival after a 48 hour treatment of microglia-neuron co-cultures with 100 ng/ml LPS in the presence or absence of FeTMPyP (2 μM), FTPPS (2 μM), MnTMPyP (5 μM), or 50 U/ml SOD and 100 U/ml catalase (SOD/CAT). Values show mean±SEM of 5 independent experiments.

FIG. 4B is a bar graph of nitrite accumulation after a 48-hour treatment of microglia-neuron co-cultures with 100 ng/ml LPS in the presence or absence of FeTMPyP (2 μM), FTPPS (2 μM), MnTMPyP (5 EM), or 50 U/ml SOD and 100 U/ml catalase (SOD/CAT). Values show mean±SEM of 5 independent experiments.

FIG. 4C is a bar graph showing the dose-dependent effect of the peroxynitrite decomposition catalyst FeTMPyP on LPS-induced microglial neurotoxicity.

FIG. 4D dose-dependent effect of the peroxynitrite decomposition catalyst FeTMPyP on LPS-induced nitrite accumulation in microglia-neuron co-cultures.

FIG. 4E is a bar graph showing the effect of SNP on neuronal survival in the presence or absence of FeTMPyP. Co-cultures are treated with LPS (100 ng/ml), SNP (300 μM), or SNP (300 μM) plus FeTMPyP (2 μM) for 48 hr. Values show mean±SEM from 5 independent experiments (a=p<0.05 compared with LPS+FeTMPyP, b=p<0.05 compared with LPS+MnTMPyP).

FIG. 4F is a bar graph showing the effect of the SNP on nitrite generation in the presence or absence of FeTMPyP. Co-cultures are treated with LPS (100 ng/ml), SNP (300 μM), or SNP (300 μM) plus FeTMPyP (2 μM) for 48 hr. Values show mean±SEM from 5 independent experiments (a=p<0.05 compared with LPS+FeTMPyP, b=p<0.05 compared with LPS+MnTMPyP).

FIG. 5A is a bar graph showing the effect on neuron survival of primary cortical neurons treated with 50 μM SIN-1 in the presence or absence of 2 μM FeTMPyP for 48 hr. Data represent mean±SEM from 3 independent experiments.

FIG. 5B is a bar graph showing the effect on nitrite accumulation in primary cortical neurons treated with 50 μM SIN-1 in the presence or absence of 2 μM FeTMPyP for 48 hr. Data represent mean±SEM from 3 independent experiments.

FIG. 5C is a bar graph showing the effect of FeTMPyP or MnTMPyP on the growth defects (ethanol) of Saccharomyces cerevisiae cells lacking cytosolic superoxide dismutase (sod1Δ). S. cerevisiae cells are inoculated in SDC medium and diluted in YPE (ethanol) medium (3 ml) containing either MnTMPyP (25 μM) or FeTMPyP (25 μM) in the presence or absence of paraquat (10 μM). Cell density is measured after 48 hours. Average of two independent experiments with duplicate samples±SEM

FIG. 6A is a bar graph showing the effect on neuron survival of a 48-hour treatment of microglia-neuron co-cultures with 5 μM Aβ₁₋₄₂ and 10 ng/ml interferon γ in the presence or absence of 1 mM L-NMMA. Mean±SEM from 4 independent experiments.

FIG. 6B is a bar graph showing the effect on nitrite accumulation of a 48-hour treatment of microglia-neuron co-cultures with 5 μM Aβ₁₋₄₂ and 10 ng/ml interferon γ in the presence or absence of 1 mM L-NMMA. Mean±SEM from 4 independent experiments.

FIG. 6C is an image of fluorescein diacetate staining of untreated neurons. The dashed arcs delineate the projection of the microglia-containing inserts (scale bar: 50 μm).

FIG. 6D is an image of fluorescein diacetate staining of neurons treated with 5 μAβ₁₋₄₂ and 10 ng/ml interferon γ. The dashed arcs delineate the projection of the microglia-containing inserts (scale bar: 50 μm).

FIG. 6E is an image of fluorescein diacetate staining of neurons treated with 5 μM Aβ₁₋₄₂, 10 ng/ml interferon γ, and 1 mM L-NMMA. The dashed arcs delineate the projection of the microglia-containing inserts (scale bar: 50 μm).

FIG. 7A is a bar graph showing the effect on neuron survival after a 48-hour treatment of microglia-neuron co-cultures with 5 μM Aβ₁₋₄₂ and 10 ng/ml interferon γ in the presence or absence of FeTMPyP (2 μM) or MnTMPyP (5 μM). Mean±SEM from 4 independent experiments (a=p<0.05 compared with Aβ; b=p<0.05 compared with Aβ+FeTMPyP).

FIG. 7B is a bar graph showing the effect on nitrite accumulation neuron survival after a 48-hour treatment of microglia-neuron co-cultures with 5 μM Aβ₁₋₄₂ and 10 ng/ml interferon γ in the presence or absence of FeTMPyP (2 μM) or MnTMPyP (5 μM). Mean±SEM from 4 independent experiments (a=p<0.05 compared with Aβ; b=p<0.05 compared with Aβ+FeTMPyP). Mean±SEM from 4 independent experiments (a=p<0.05 compared with Aβ; b=p<0.05 compared with Aβ+FeTMPyP).

FIGS. 8A and 8B are images of TdT-mediate dUTP nick end labeling (TUNEL) in neurons removed from microglia-neuron co-cultures treated with 5 μM Aβ₁₋₄₂+10 ng/ml interferon γ for 24 hr. Whereas neurons exposed to inactive microglia are scarcely stained (FIG. 8A) (white arrowheads), neurons exposed to Aβ₁₋₄₂-activated microglia (FIG. 8B), are predominantly positively stained (black arrowheads) (scale bar: 20 μM).

FIG. 8C is the percentage of TUNEL-negative neurons and surviving neurons after 24 hour treatment of microglia-neurons co-cultures with 5 μM Aβ₁₋₄₂ plus 10 ng/mL interferon γ.

### DETAILED DESCRIPTION OF THE INVENTION

The present invention is based on the finding that peroxynitrite, generated from the reaction of nitric oxide and superoxide produced by activated microglia, is the major mediator of Aβ neurotoxicity. This is based in part on the discovery of compounds that have little or no effect on nitric oxide generation can still increase neuron survival. This protection is due to the scavenging of peroxynitrite produced by the nitric oxide, and superoxide generated by activated microglia.

More specifically, it was discovered that compounds that decompose or scavenge peroxynitrite, without affecting the normal activity of microglia, are sufficient to treat neurogenerative diseases caused by activated microglia. That is, in one embodiment of this invention, compounds that only scavenge peroxynitrite, and do not need to affect the concentration of other neurotoxins, are sufficient to alleviate the pathology of conditions affected by the presence of peroxynitrite.

Accordingly, one aspect of this invention provides a method of treating a medical condition in a subject, wherein said condition is affected by the presence of peroxynitrite, said method comprising administering to the subject a compound that decomposes peroxynitrite but is not known to negatively affect the normal activity of microglia and other brain cells such as neurons, astrocytes, and oligodendrocytes, wherein the decomposition of peroxynitrite alone is sufficient to alleviate the pathology of said condition.

As used herein, the phrase “normal activity of microglia and other brain cells” includes any activity of microglia which is beneficial to the subject, such as secreting neuroprotective molecules, clearing Aβ, and supporting other normal brain functions as well as the normal activities of other cells in the brain.

This invention further provides a method of treating a medical condition in a subject, wherein said condition is affected by activated microglia, said method comprising administering to the subject a compound that decomposes peroxynitrite but does not negatively affect the normal activity of microglia and other brain cells, wherein the decomposition of peroxynitrite alone is sufficient to alleviate the pathology of said condition.

As used herein, the term “activated microglia” includes, but is not limited to, microglia activated by amyloid fi-peptide (Aβ)₁₋₄₂, anti-Aβ-antibodies, a combination of Aβ₁₋₄₂ and anti-Aβ-antibodies, or lipopolysaccharide (LPS).

It was further discovered that compounds inhibit TNF-α secretion from activated microglia, without affecting the normal activity of microglia, are sufficient to treat neurogencrative diseases caused by activated microglia. That is, according to another embodiment of this invention, compounds that only inhibit TNF-α secretion, and do not need to affect the concentration of other neurotoxins, are sufficient to alleviate the pathology of conditions affected by the presence of TNF-α. Another aspect of this invention provides a method of treating a medical condition in a subject, wherein said condition is affected by the presence of TNF-α, said method comprising administering to the subject a compound that inhibits secretion of TNF-α from microglia but does not negatively affect the normal activity of microglia and other brain cells wherein the inhibition of TNF-α secretion alone is sufficient to alleviate the pathology of said condition.

Medical conditions that can be treated according to the methods of this invention include, but are not limited to, conditions caused by Aβ or LPS-activated microglia or diseases caused by the presence of peroxynitrite or the secretion of TNF-α from microglia. A non-limiting example of a medical condition that can be treated according to this invention is Alzheimer's disease.

It is known [Smith, 2002] that certain vaccines for Alzheimer's disease increase microglial activity of clearing Aβ by generating oxidants such as nitric oxide, which in turn generates peroxynitrite. As discussed above, it was discovered by the present inventors that it is desirable to maintain normal activity of microglia while scavenging peroxynitrite formed as a result of microglial activation in the treatment of conditions caused by the presence of such neurotoxin. Accordingly, another embodiment of this invention comprises a method of treating a medical condition in a subject, wherein said condition is affected by the presence of peroxynitrite, said method comprising administering to the subject a vaccine that increases microglia activity of clearing Aβ, wherein the method further comprises administering a compound that decomposes peroxynitrite but does not negatively affect the normal activity of microglia and other brain cells, wherein the decomposition of peroxynitrite alone is sufficient to alleviate the pathology of said condition. The compound that decomposes peroxynitrite may be added before, concurrently with, or after administration of the vaccine. An example of a suitable vaccine includes that disclosed by Marwick [2000], which is incorporated herein by reference.

The present invention utilizes a co-culture system in which neurons are co-incubated with activated microglia which generate peroxynitrite for many hours during the treatment. In contrast, the media removed from activated microglia and transferred to neuronal cultures in experiments performed by other [Combs, 2001] contain very little of the short-lived peroxynitrite. Thus, the low dose of Aβ₁₋₄₂ (5 μM) used in the experiments of the present invention induces both peroxynitrite and TNF-α generation, but high levels of peroxynitrite mediate the majority of the toxicity during acute treatment. In contrast, higher doses of Aβ or longer treatments may induce neurotoxic levels of TNF-α. This may explain the role of both NO and TNF-α in the neurotoxicity of microglia treated with Aβ₁₋₄₂ or Aβ₂₅₋₃₅ in which neurons and microglia were co-cultured and were treated with a higher concentration of Aβ₁₋₄₂ (12 μM) for a longer time (72 hours) than in our experiments [Meda, 1995].

Neurotoxicity according to the methods of this invention is studied in a co-cultures system in which microglia and neurons can be separated before cell death analysis. Accordingly, this invention further provides a method of screening an effective test compound that decreases neuron death caused by a neurotoxin, said method comprising:

(a) providing a co-culture of microglia and neurons;

(b) exposing said co-culture to said test compound to form a test mixture;

(c) subjecting said test mixture to conditions that activate said microglia;

(d) examining said test mixture at a selected time after said subjection for the extent of neuron cell death; and

(e) measuring the extent of neuron death, wherein said effective test compound is identified as a compound that decreases neuron death relative to a control sample and does not affect normal activity of microglia.

Compounds that scavenge or decompose peroxynitrite or inhibit TNF-α secretion from microglia may be tested for efficacy according to the methods of this invention using assays described below, i.e., in assays for nitrite accumulation as an indirect measurement of nitric oxide production, in assays for superoxide generation, in assays for NO synthase inhibition; in assays for peroxynitrite inhibition; in assays for TNF-α secretion, or in assays for neuronal cell death by TUNEL staining (see below). Compounds most preferred are those which effect the greatest protection of neurons from peroxynitrite or TNF-α generated from Aβ- or LPS-activated microglia.

LPS-Dependent Neuronal Death Follows the Generation of Superoxide/Peroxynitrite and NO/Nitrite by Microglia.

Microglia, the resident macrophages of the central nervous system, upon activation can produce large quantity of nitric oxide synthesized by inducible nitric oxide synthase (iNOS). Activated microglia also produce abundant peroxide through the membrane-associated NADPH-oxidase.

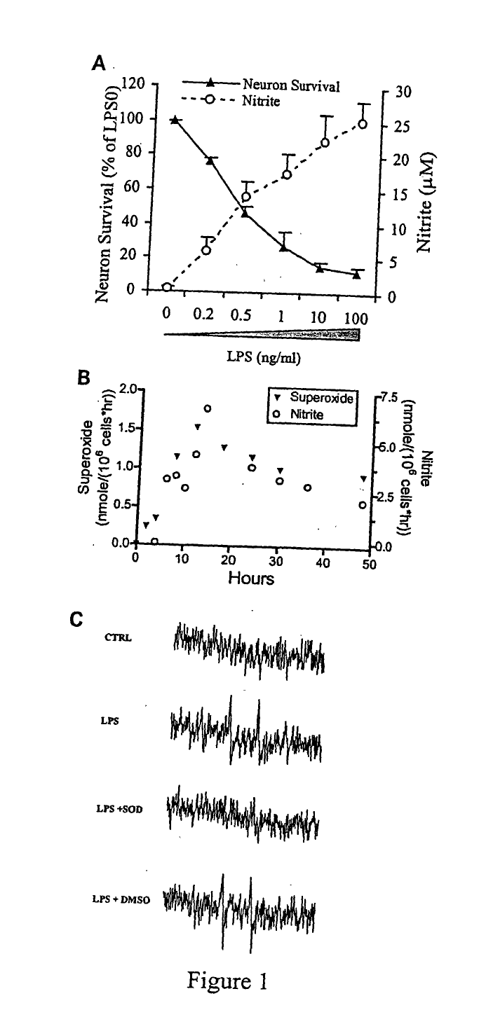

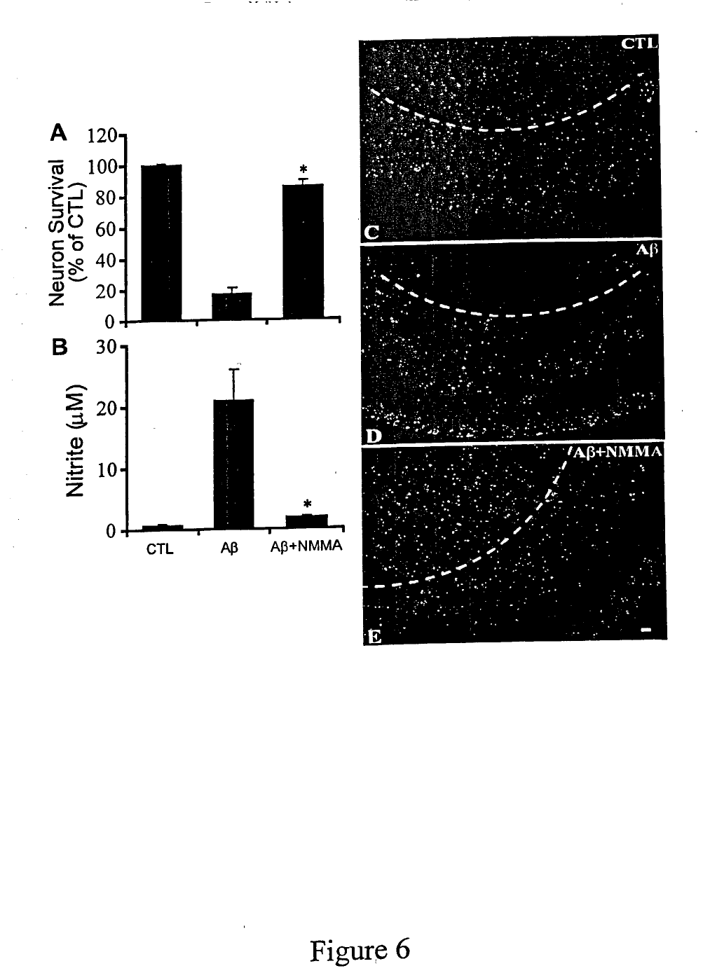

FIG. 1A shows that LPS induces dose-dependent nitrite generation and neuronal death in microglia-neuron co-cultures after a 48 hour treatment with 0.2 to 100 ng/ml LPS. LPS-activated microglia caused the death of 20% of neurons by 24 hours, 50% at 36 hours and 80% at 48 hours. Only the neurons directly under the insert (Costar, membrane pore size 0.4 μM) containing the microglia (1 mm distance) were killed, suggesting that neurotoxicity is mediated by short-lived diffusible molecules (see FIG. 6D).

As a first step in determining whether neurotoxicity correlated with nitric oxide generation, the time course of nitric oxide and superoxide generation was investigated. Nitric oxide generation was assayed by measuring the concentration of nitrite, a metabolite of nitric oxide, released into the medium [Ding, 1988]. As shown in FIG. 1B, 100 ng/ml of LPS was observed to induce nitrite generation beginning at 6 hours (reported as μM generated/hour), with a peak at approximately 14 hours and a gradual decline until 48 hours. Similarly to NO, superoxide generation peaked at 12 hours as also shown in FIG. 1B.

To monitor the generation of superoxide by activated microglia, Electron Paramagnetic Resonance (EPR) measurements were performed with 5,5-dimethyl-1-pyrroline-N-oxide (DMPO) as a superoxide spin trap. The scans are shown in FIG. 1C. To confirm that the EPR signal is caused by superoxide, either superoxide dismutase (SOD) or DMSO, which competes with DMPO for hydroxyl radical, were added to the microglia cells together with LPS. The absence of a signal in the presence of SOD (third trace) together with the similarities of the traces in the presence or absence of DMSO (trace 2 and 4) indicates that the EPR signal is caused by superoxide and not by hydroxyl radical.

The data presented herein show that in microglia exposed to LPS, the generation of NO reaches its peak after 10-20 hours and continues for over 50 hours, whereas the generation of toxic oxygen species including peroxynitrite and superoxide increases sharply at 24 hours to reach peak levels after 30-50 hours. These results demonstrate that LPS-activated microglia kill neurons in a dose-dependent manner.

Nitric Oxide is Required for the Neurotoxicity of Activated Microglia.

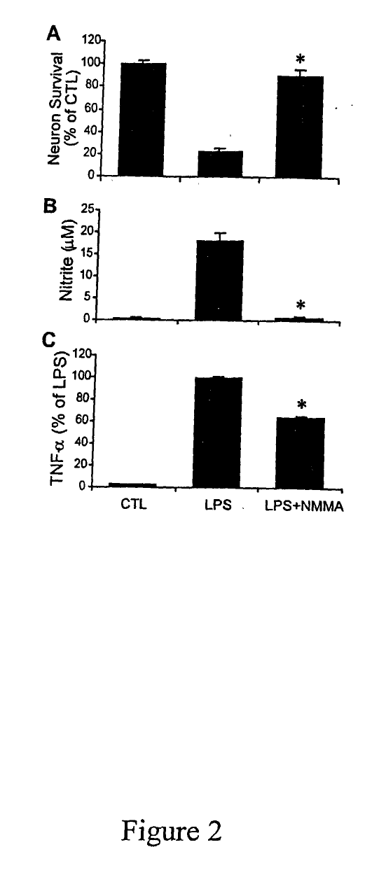

It has been reported that the inhibition of iNOS by NMMA blocks NO generation and the toxicity of microglia activated with both LPS and Aβ₁₋₄₂ [Ii, 1996]. In the present invention, the NO synthase inhibitor L-NMMA was used to test whether nitric oxide mediates neuron-killing by activated microglia. As shown in FIGS. 2A-2C, when L-NMMA was added at the same time with LPS, L-NMMA completely blocked LPS-induced neuron death (FIG. 2A; viable neurons 92.4±7.4% of control) and NO synthesis (FIG. 2B), and reduced TNF-α secretion by 35% (FIG. 2C). This data suggests that the neurotoxicity of LPS-activated microglia is nitric oxide-dependent, and that inhibition of NO synthesis is sufficient to completely block the neurotoxicity of activated microglia.

It is known that TNF-α, a cytokine released by activated microglia, can be both neurotoxic [Chao, 1993; Wood, 1995] and neuroprotective [Barger, 1995]. It has also been reported that pentoxifylline is a non-selective phosphodiesterase inhibitor that blocks the release of TNF-α from microglia [Chao, 1992].

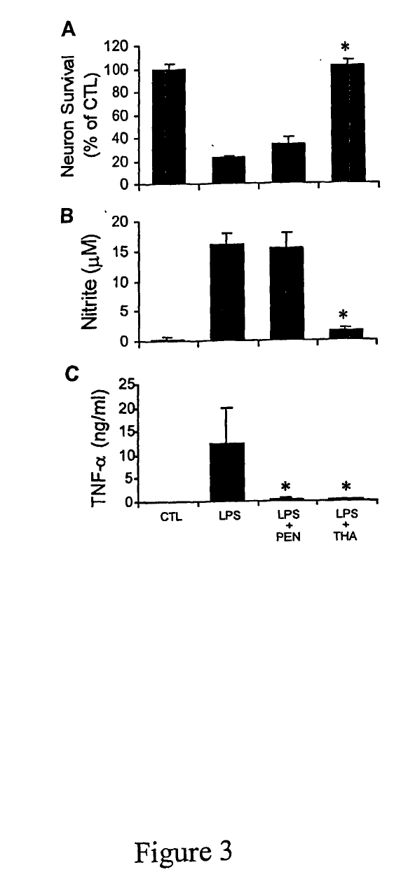

Thus, in a further embodiment of this invention, two inhibitors of TNF-α production, i.e., pentoxifylline (PEN) and thalidomide (THA), were used to test whether TNF-α mediates neuron-killing by activated microglia. Microglia-neuron co-cultures were treated with 100 ng/ml LPS for 48 hours alone or together with either the TNF-α inhibitor pentoxifylline (PEN, 500 μM) or the TNF-α inhibitor thalidomide (THA, 200 μM). FIGS. 3A-C show that pentoxifylline inhibits TNF-α secretion (FIG. 3C) but has little effect on nitrite accumulation (FIG. 3B) and neuron killing (FIG. 3A). In contrast, thalidomide, another TNF-α inhibitor that inhibits NO production, completely blocked neuron death (FIG. 3A) and inhibited both NO production (FIG. 3B) and TNF-α secretion (FIG. 3C).

Peroxynitrite Mediates the Neurotoxicity of Microglia Activated by LPS.

Nitric oxide reacts with superoxide (O₂.⁻) at a near diffusion-limit speed with a rate constant of 6.7×10⁹ M⁻¹ sec⁻¹, producing the highly reactive and toxic peroxynitrite (ONOO⁻) [Ischiropoulos, 1992]. Furthermore, unlike superoxide, peroxynitrite is membrane-permeable [Marla, 1997], and is able to reach and damage neuronal DNA, lipids and proteins [Estevez, Prog. Brain Res., 1998; Estevez, J. Neurosci., 1998]. Since activated microglia generate both NO and superoxide, peroxynitrite is hypothesized to be a major mediator in microglia induced neuronal injury [Combs, 2001; Van Dyke, 1997].

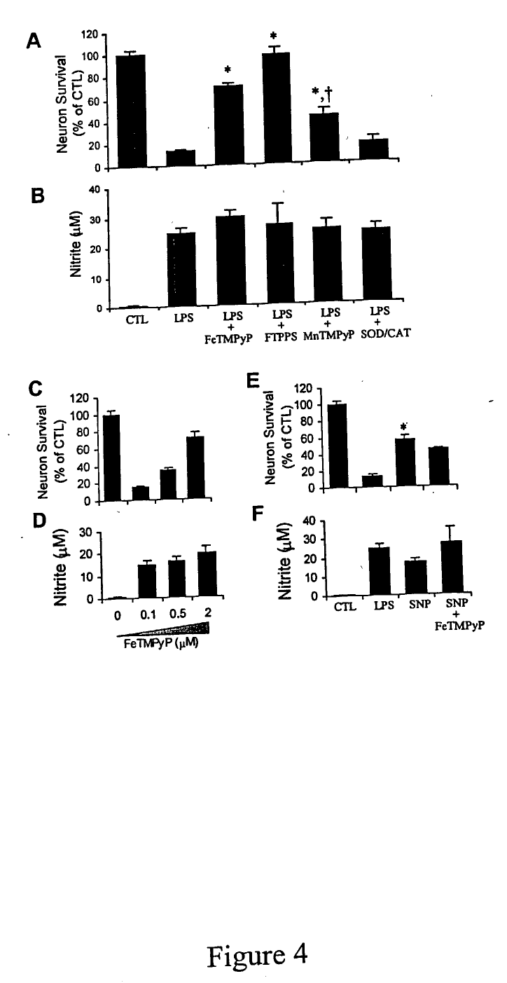

To test the role of superoxide and peroxynitrite in the toxicity of activated microglia we treated neurons/microglia co-cultures with the 100 ng/ml LPS in the presence or absence of: 2 μM of the peroxynitrite decomposition catalyst FeTMPyP, 2 μM of the peroxynitrite decomposition catalyst FeTPPS, 5 μM of the superoxide dismutase mimetic MnTMPyP, or 50 U/ml SOD+100 U/ml catalase (SOD/CAT). It was observed that the membrane-permeable iron porphyrin peroxynitrite decomposition catalysts FeTMPyP and FeTPPS blocked LPS-induced microglia neurotoxicity (FIG. 4A) without decreasing NO production (FIG. 4B).

As shown by the results in FIGS. 4C and 4D, the protective action of FeTMPyP is dose-dependent with an optimal concentration of 2 μM. FIG. 4C shows the dose-dependent effect of the peroxynitrite decomposition catalyst FeTMPyP on LPS-induced microglial neurotoxicity. FIG. 4D shows dose-dependent effect of the peroxynitrite decomposition catalyst FeTMPyP on LPS-induced nitrite accumulation in microglia-neuron co-cultures.

To test whether peroxynitrite is formed in neurons (instead of microglia) from the reaction of exogenous nitric oxide with neuronal superoxide, cells were treated with a concentration of the NO donor sodium nitroprusside (SNP, 300 μM), which generates nitrite/NO at levels similar to those generated by LPS-activated microglia. Co-cultures treated in the presence or absence of the peroxynitrite decomposition catalyst FeTMPyP (2 μM) for 48 hr. The results are summarized in FIGS. 4E-4F. At 300 μM, SNP killed less than 50% of the neurons, significantly below the 85% cell death caused by LPS-induced activation of microglia (FIG. 4E). Furthermore, SNP-induced neuron death could not be blocked by the peroxynitrite decomposition catalyst FeTMPyP (FIG. 4E), confirming that SNP toxicity is caused by NO and not peroxynitrite. These results suggest that the superoxide and peroxynitrite that mediate the neurotoxicity of microglia activated by LPS are generated by the microglia and not by the neurons.

Since superoxide anion is required to form peroxynitrite, superoxide dismutation should attenuate peroxynitrite production and decrease neuron death. We tested the protective effect of the manganese porphyrin SOD mimetic MnTMPyP (membrane-permeable) in the co-culture treated with LPS. MnTMPyP attenuated LPS-induced microglial neurotoxicity without compromising nitric oxide production (FIGS. 4A and 4B). In contrast, treatment with SOD plus catalase, or each scavenger alone (data not shown); did not protect against microglial neurotoxicity (FIGS. 4A and 4B) suggesting that peroxynitrite is generated inside microglia, where superoxide cannot be reached by the non-membrane permeable SOD. Because of its short half-life (1.9 sec at pH 7.4), the released peroxynitrite reaches only the neurons cultured directly under the insert containing the microglia (see FIG. 6D). The inability of catalase to block neuronal death shows that hydrogen peroxide is not a major mediator of microglial neurotoxicity.

The inhibition of cell death by FeTMPyP suggests that peroxynitrite, rather than NO, is the main mediator of the neurotoxicity of LPS and Aβ₁₋₄₂. The high second order reaction rate constant between peroxynitrite and FeTMPyP (5×10⁷ M⁻¹s⁻¹) enables this efficient decomposition catalyst to protect mammalian cells against high doses of peroxynitrite [Salvemini, 1998].

The data in FIGS. 2C and 3C suggest that TNF-α is a minor but significant mediator of the toxicity of activated microglia in part base on the following: a) the partial or total inhibition of TNF-α secretion by NMMA or thalidomide, in addition to the inhibition of NO generation, was associated with the complete inhibition of microglial toxicity, as shown in FIGS. 3A-3C. In contrast, 20-30% of the neurons exposed to activated microglia died in the presence of the peroxynitrite decomposition catalysts, whereas about 50% of neurons died in the presence of the superoxide mimetic (see FIGS. 4A and 4B); and b) the inhibition of TNF-α secretion in pentoxifylline-treated co-cultures, which did not decrease nitrite/NO generation, improved the survival of neurons exposed to microglia activated by LPS. Peroxynitrite is a short-lived molecule with a half-life of less than two seconds, whereas TNF-α is a relatively long-lived protein. Therefore the pattern of cell death in neurons confined to an area within a few millimeters of the microglia (see FIG. 6D) does not indicate a major role for TNF-α in acute neurotoxicity. However, TNF-α may be more toxic at higher concentrations, or during longer or chronic treatments. In fact, it has been reported that TNF-α contained in media removed from monocytes and microglia stimulated with a higher concentration of Aβ₁₋₄₀ or Aβ₂₅₋₃₅ (60 μM) causes neuronal apoptosis [Combs, 2001].

FeTMPyP is an Efficient Decomposition Catalyst of Peroxynitrite but not Superoxide.

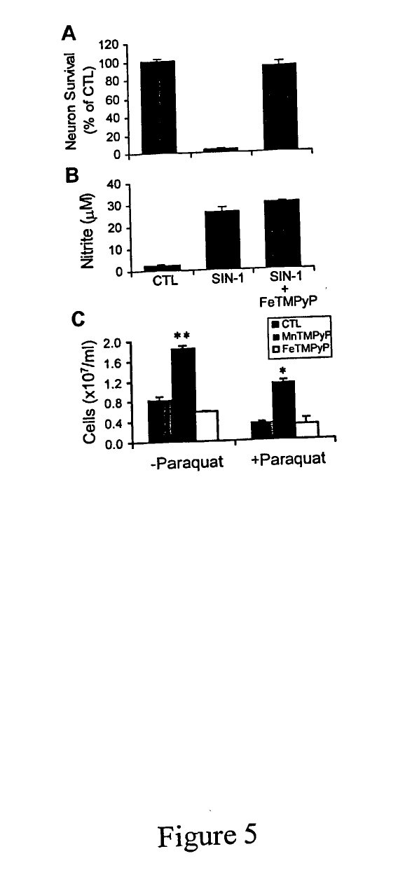

To validate the specificity and effectiveness of the peroxynitrite decomposition catalyst FeTMPyP, this catalyst was further tested with neuron cultures treated with SIN 1, an exogenous NO and superoxide donor that, consequently, generates peroxynitrite under physiological conditions [Hogg, 1992]. Primary cortical neurons were treated with 50 μM SIN-1 in the presence or absence of 2 μM FeTMPyP for 48 hr. As shown in FIG. 5A, FeTMPyP effectively protected neurons against SIN-1 induced peroxynitrite toxicity. The effect of FeTMPyP on nitrite accumulation is shown in FIG. 5B.

To further test the specificity of FeTMPyP and determine whether it may be protecting neurons by also scavenging superoxide, its effect on yeast mutants lacing cytosolic SOD (sod1Δ) was studied. This is a valuable system to test the specificity of these agents because the primary cause of all the defects of yeast sod1Δ mutants is superoxide toxicity. By contrast, molecules like paraquat and menadione generate other toxic oxygen species in addition to superoxide intracellularly. FIG. 5C shows the effect of FeTMPyP or MnTMPyP on the growth defects (ethanol) of Saccharomyces cerevisiae cells lacking cytosolic superoxide dismutase (sod1Δ). S. cerevisiae cells were inoculated in SDC medium and diluted in YPE (ethanol) medium (3 ml) containing MnTMPyP (25 μM) or FeTMPyP (25 μM) in the presence or absence of paraquat (10 μM). Cell density was measured after 48 hours. FeTMPyP did not reverse the growth defects of sod1Δ mutants either in the absence or presence of paraquat. By contrast the superoxide scavenger MnTMPyP blocked superoxide toxicity and reversed the growth defects of sod1Δ mutants whether or not paraquat was present. These results confirm that FeTMPyP is acting as a specific and efficient decomposition catalyst of peroxynitrite.

However, the SOD mimetic MnTMPyP functions as a permeable superoxide dismutase and partially blocked the neurotoxicity of microglia, which raised the possibility that the protective role of FeTMPyP may be caused by its reaction with superoxide. This possibility was ruled out, as demonstrated above, by showing that FeTMPyP did not reverse the defects of yeast lacking either cytosolic or mitochondrail SODs (FIG. 5C, and data not shown). The effectiveness of FeTMPyP in blocking peroxynitrite toxicity was also confirmed by its ability to protect neurons against SIN-1, which generates peroxynitrite (FIGS. 5A and 5B).

Peroxynitrite Mediates the Neurotoxicity of Microglia Activated by Aβ.

To test whether peroxynitrite may also play a role in the toxicity of the amyloid β peptide (Aβ) associated with Alzheimer's disease, the role of agents that block or scavenge specific nitrogen and oxygen species in protecting neurons against fibrillar Aβ₁₋₄₂ was tested. Microglianeuron co-cultures were treated with (1) 5 μM Aβ₁₋₄₂ and 10 ng/ml interferon γ in the presence or absence of the NOS inhibitor L-NMMA. FIG. 6A shows the effect on neuron survival, and FIG. 6B shows the effect on nitrite accumulation. A 48-hour treatment of microglia neuron co-cultures with 5 μM Aβ₁₋₄₂+10 ng/ml interferon γ without L-NNMA resulted in the death of over 80% of neurons and required the generation of nitric oxide by microglia as was shown previously [Ii, 1996]. Treatment of neurons with 5 μM Aβ₁₋₄₂ did not result in neurotoxicity in the absence of microglia (data not shown).

FIGS. 6C-E show fluorescein diacetate staining of untreated neurons (FIG. 6C), neurons treated with 5 μAβ₁₋₄₂+10 ng/ml interferon γ (FIG. 6D), and neurons treated with 5 μM Aβ₁₋₄₂+10 ng/ml interferon γ+1 mM NOS inhibitor L-NMMA (FIG. 6E). Dashed arcs delineate the projection of the microglia-containing inserts. Cell death was confined to the region directly underneath the inserts containing microglia, in an area slightly larger (15%) than that of the insert, as shown in FIG. 6D. The dark region in the upper portion of the figure indicating dead cells primarily in the region directly underneath the microglia-containing culture inserts (see Example 2), in contrast to the homogenous staining of untreated neurons shown in FIG. 6C. The spatial proximity of dead neurons to activated microglia (<1 mm) is consistent with the role of a short-lived molecule, peroxynitrite, in the mediation of Aβ₁₋₄₂ neurotoxicity. These results show that the neurotoxicity of Aβ₁₋₄₂-activated microglia is nitric oxide-dependent.

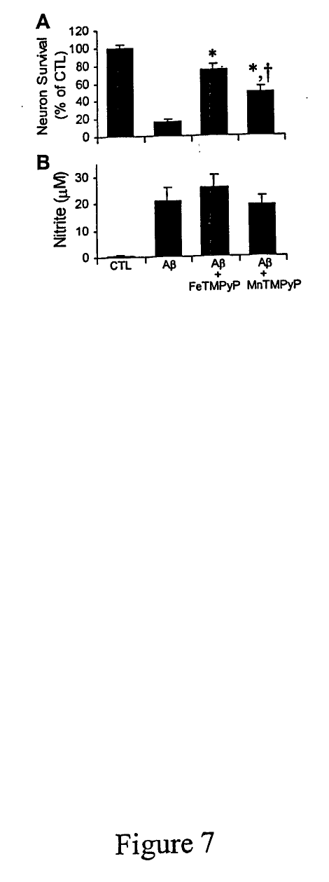

FIGS. 7A and 7B show the effects on neuron survival and nitrite accumulation, respectively, after a 48 hour treatment of microglia-neuron co-cultures with 5 μM Aβ₁₋₄₂ and 10 ng/ml interferon γ in the presence or absence of the peroxynitrite decomposition catalyst FeTMPyP (2 μM) or the SOD mimetic MnTMPyP (5 μM). The peroxynitrite decomposition catalyst FeTMPyP also blocked AP-induced microglial neurotoxicity without compromising nitric oxide production. The SOD mimetic MnTMPyP had a similar but attenuated effect. These results suggest that peroxynitrite is also a major mediator the toxicity of microglia activated by Aβ.

Aβ₁₋₄₂ Causes Peroxynitrite-Dependent DNA Fragmentation Preceding Cell Death.

Whereas high concentrations of peroxynitrite are known to induce necrotic cell death in neurons, low levels of peroxynitrite can induces apoptosis [Bonfoco, 1995; Estevez, 1998]. The fragmentation of genomic DNA following the internucleosoal cleavage during apoptosis is a widely used marker for apoptosis.

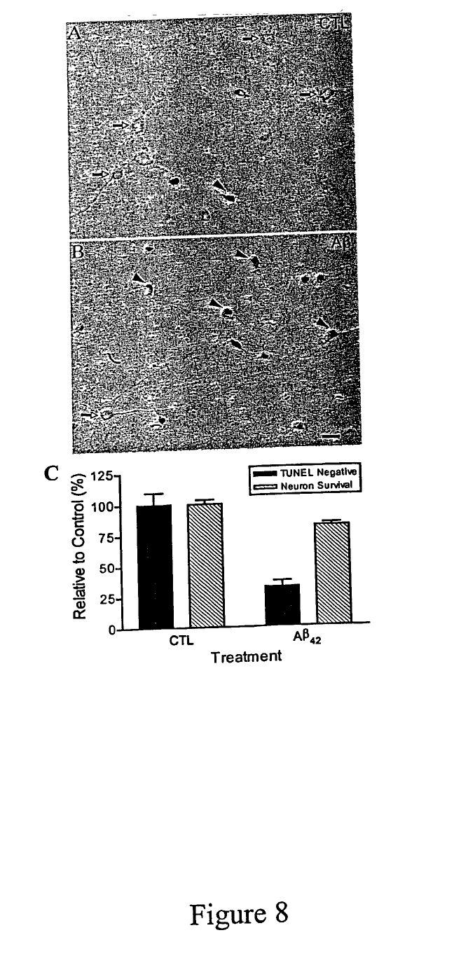

FIGS. 8A and 8B show images of Aβ₁₋₄₂-activated microglia induce TUNEL staining preceding loss of viability. DNA fragmentation was detected in vivo by the TdT-mediated dUTP nick end labeling (TUNEL) in neurons removed from microglia-neuron co-cultures treated with 5 μM Aβ₁₋₄₂ and 10 ng/ml interferon γ for 24 hours. Whereas neurons exposed to inactive microglia are scarcely stained (FIG. 8A) (white arrowheads), neurons exposed to Aβ₁₋₄₂ activated microglia (FIG. 8B), are predominantly positively stained (black arrowheads), scale bar 20 μM. As shown in FIG. 8B, the majority of the neurons treated with Aβ₁₋₄₂ were TUNEL-labeled at 24 hours, suggesting that peroxynitrite induces apoptosis. DNA strand breaks were observed at 24 hours in Aβ-treated neurons in the area below the microglial inserts, when less than 30% of the cells are dead (data not shown). Although TUNEL assay can label both apoptotic and necrotic neurons [Adamec, 2001], most of TUNEL-labeled cells at 24 hours were not necrotic as determined by the fluorescein diacetate staining (FIGS. 6A-6C). These results are consistent with the demonstrated role of peroxynitrite in inducing apoptosis in motor neurons [Estevez, 1998]. The time course of oxidant-generation shown in FIGS. 1A and 1B may explain the DNA fragmentation observed at 24 hours, followed by cell death at 48 hr treatment (FIGS. 8A and 8B).

The data presented in this invention show that cell death in microglia-neurons co-cultures exposed to LPS or Aβ₁₋₄₂ occurred only in neurons separated from microglia by 1 mm, and was prevented by compounds that block NO synthesis or scavenge peroxynitrite, and to a lesser extent superoxide. Taken together, these results suggest that the short-lived peroxynitrite generated by activated microglia is the major mediator of neurotoxicity during acute treatment of microglia and neurons with Aβ₁₋₄₂ or LPS. In contrast, TNF-α appears to play a minor but significant role in the toxicity of activated microglia.

A role for peroxynitrite in the toxicity of Aβ or activated microglia, as shown by the present invention, is consistent with the increase in protein nitration in Alzheimer's disease brain tissues, but not in age-matched control brains. Together, these findings suggest that peroxynitrite generation may play a major role in Alzheimer's disease [Smith, 1997].

That peroxynitrite is the major mediator of the toxicity of microglia activated by either Aβ or LPS is supported by several results presented herein, including: a) the neurotoxicity of Aβ is confined to the area directly exposed to microglia, b) the peak of DCF fluorescence, induced by peroxynitrite and superoxide, and not that of nitrite/NO generation, coincides with neurotoxicity, c) the NO synthase inhibitor NMMA blocks toxicity as previously reported [Ii, 1996], d) FeTMPyP scavenges peroxynitrite, but not NO or superoxide, and blocks the toxicity of microglia activated by LPS or Aβ₁₋₄₂, and e) the superoxide mimetic MnTMPyP, which decreases peroxynitrite generation, partially blocks the toxicity of activated microglia.

In summary, the results presented herein using microglia-neuron co-culture models show that peroxynitrite is the major acute mediator of the neurotoxicity of microglia activated by LPS or Aβ. In addition TNF-α contributes to neurotoxicity and, because of its sustained activity, may also play an important role in the toxicity of the chronically activated microglia of Alzheimer's disease brains. These findings support the development of drugs that protect neurons against the toxicity of specific molecules such as peroxynitrite and TNF-α without interfering with the normal functions of microglia.

The invention is further illustrated by the following non-limited examples. All scientific and technical terms have the meanings as understood by one with ordinary skill in the art. The descriptions and specific examples that follow are only intended for the purposes of illustration, and are not to be construed as limiting in any manner to isolated the compounds of the present invention by other methods. Further, variations of the methods to produce the same compounds in somewhat different fashion will be evident to one skilled in the art.

Lipopolysaccharide (LPS, E. coli strain O26:B6), superoxide dimutase (SOD), catalase, sodium nitroprusside (SNP), and fluoresceindiacetate were obtained from Sigma (St. Louis, Mo.). Recombinant mouse interferon γ was obtained from R&D Systems (Minneapolis, Minn.). N^{G}-Monomethyl-L-arginine (L-NMMA), 3-morpholinosydnonimine (SIN-1), 5,10,15,20-Tetrakis(4-sulfonatophenyl)prophyrinato iron (III) chloride (FTPPS), 5,10,15,20-Tetrakis(N methyl-4′-pyridyl)porphinato iron (III) chloride (FeTMPyP), and Mn(III)tetrakis(1-methyl-4-pyridyl)porphyrin pentachloride (MnTMPyP) were obtained from Calbiochem (San Diego, Calif.). Thalidomide and pentoxifylline were obtained from RBI (Natick, Mass.). All reagents were tested to be not neurotoxic at the concentrations applied in neuron cultures.

Aβ₁₋₄₂ (US peptide, Fullerton, Calif.) was dissolved in DMSO (Sigma) to obtain a 5 mM stock and kept at −70° C. Before treating the cultures, a 50 μM Aβ₁₋₄₂ solution was prepared in F12K medium as 10×solution and incubated at 37° C. for one day to obtain aggregated Aβ. For all treatments with Aβ₁₋₄₂, 10 ng/ml interferon γ was also added as a priming factor (Meda et al., 1995; Ii et al., 1996).

The invention may be better understood with reference to the accompanying examples that are intended for purposes of illustration only and should not be construed as, in any sense, limiting the scope of the present invention, as defined in the claims appended hereto. While the described procedures in the following examples are typical of those that might be used, other procedures known to those skilled in the art may alternatively be utilized. Indeed, those of ordinary skill in the art can readily envision and produce further embodiments, based on the teachings herein, without undue experimentation.

### EXAMPLE I

### Cell Culture

Rat primary glial cells were derived from cerebral cortices of neonatal (postnatal day 3) Fisher 344 rat (Giulian and Baker, 1986). Dispersed cells were grown in Dulbeco's modified Eagle's medium. (DMEM)/F12 (Cellgro, Mediatech, Hermdon, Va.) supplemented with 10% heat-inactivated fetal bovine serum (FBS, HyClone Laboratories, Logan, Utah), 50 U/ml penicillin (Sigma, St. Louis, Mo.), and 0.05 mg/ml streptomycin (Sigma), at 37° C. in a humidified 95%/5% (v/v) mixture of air and CO₂. Culture media were renewed twice a week. After 14-21 days in culture, microglia were detached from the monolayer by gentle shaking and replated into cell culture inserts (Costar, Corning Inc., Corning, N.Y.) or 96-well (3×10⁴ cells/well) cell culture plates (Falcon, Becton Dickinson Labware, Franklin Lakes, N.J.). The microglia homogeneity achieved by this procedure was>98%, as determined by immunocytochemistry for microglial marker complement receptor type 3 (CR3) using mouse anti-rat CR3 antibody OX42 (Serotec, Raleigh, N.C.; dilution 1:50) (Morgan et al, 1995).

Neuron cultures were derived from fatal (embryonic day 17) Fishes 344 rat cerebral cortices detailed previously (Banker and Goslin, 1988; Rozovsky et al., 1994) and plated at 5×10⁴ viable cells/well is poly-D-lysine (Sigma) coated 24-well plates (Costar). Culture media were renewed after 1 hour and not changed until the time of experiment at 6-7 days in culture. Microglia were harvested from mixed-glia cultures, plated in 9 mm cell culture insects (Costar, membrane pore size 0.4 μm) at 10 cells/insert, and placed into the culture wells containing neurons. The porous membrane allows free diffusion of molecules. The distance between neuron layer on the culture plate and microglia layer on the insert membrane is 1 mm, according to manufacturer's description. Treatment started 3 to 4 hours afterwards. Neuron-microglia cocultures were maintained in glial medium as described above.

### EXAMPLE 2

### Neuron Viability Assay

Following treatment, culture inserts containing microglia were removed and neurons were stained with 10 jig/ml fluorescein diacetate (FDA, Sigma) for 10 min. FDA is membrane-permeable and freely enters intact cells where it is hydrolyzed by cytosolic esterase and converted to membrane-impermeable fluorescein with a green fluorescence, exhibited only by live cells. Since neuron deaths occur primarily in the region directly underneath the microglia-containing culture inserts (see FIG. 6D), for quantification, eight images at the center of each well were taken with a Nikon TE300 fluorescent microscope and analyzed with the IP Lab imaging software (version 3.54, Scanalytics, Fairfax, Va.). Viable neurons were quantified by the area covered by green fluorescence, after the establishment of a linear relationship between the numbers of stained cells and the green fluorescent area. The total area analyzed occupied 30% of the area where neuron death occurred.

### EXAMPLE 3

### Nitrite Measurement

Nitric oxide (NO) production was determined indirectly through the assay of nitrite (NO₂), a stable metabolite of NO, based on the Griess reaction (Huygen, 1970; Green et al., 1982; Ding et al., 1988). Briefly, a 50 μl aliquot of conditioned media was mixed with an equal volume of Griess reagent (0.1% N-(1-naphthyl)ethylenediamine dihydrochloride, 1% sufanilamide, and 2.5% phosphoric acid, all from Sigma), and incubated for 10 min at 22° C. The absorbance was read at 550 nm on a microtiter plate reader (Spectra MAX 250, Molecular Devices, Sunnyvale, Calif.). Nitrite concentrations were calculated from a standard curve of NaNO₂ (Sigma) ranging from 0 to 100 μM. Background NO₂ was subtracted from the experimental values.

### EXAMPLE 4

### Detection of Superoxide/Peroxynitrite by Electron Paramagnetic Resonance (EPR)

For EPR measurements, microglia cells (100,000) with or without LPS treatment were incubated in 200 μl culture medium containing 120 mm DMPO. After 15 minutes, the medium was removed and analyzed by EPR. EPR spectra were recorded on a Bruker ECS106 spectrometer with the following settings: receiver gain: 5×10⁵; microwave power: 20 mW; microwave frequency: 9.81 GHz; modulation amplitude: 1 G; time constant: 1.3 seconds; scan time: 87 seconds; scan width: 80 G. The DMPO-OH signal generated from heart mitochondria were quantified by comparison with TEMPOL standard after doubly integration of both signals. All scans shown are an accumulation of 7 scans.

### EXAMPLE 5

### TNF-α Elisa

Levels of secreted TNF-α in culture supernatants were determined by an enzyme-linked immuno-sorbent assay (Elisa) kit following manufacturer's instructions (BioSource International, Camarillo, Calif.).

### EXAMPLE 6

### Yeast Sod Mutants and Growth Assay

EG118 (sod1Δ) (DBY746 wt with sodl::URA3) yeast were grown in liquid media in SDC-synthetic complete medium with 2% glucose, supplemented with amino acids, adenine, uracil as well as a four-fold excess of the supplements tryptophan, leucine, histidine, lysine and methionine (Longo et al., 1996). Overnight cultures were gown in selective media and inoculated with a flask volume/medium volume ratio of 5:1 at 30° C. with shaking at 220 rpm. After overnight cultures in SDC medium sod1Δ cells were diluted to an Optical Density at 600 nm (OD₆₀₀) of 0.1 and inoculated in 3 ml of YPE medium (2% ethanol) containing MnTMPyP (25 μM) or FeTMPyP (25 μM)+/−paraquat (10 μM). Call density was determined after 48 hr by OD.

### EXAMPLE 7

### TUNEL Staining

DNA cleavage in apoptotic nuclei was detected with In Situ Cell Death Detection Kit as described by the manufacturer (Roche Molecular Biochemicals, Indianapolis, Ind.). Briefly, cells were fixed with paraformaldehyde (4% in PBS, pH 7.4) and permeablized (0.1% Triton X-100 in 0.1% sodium citrate). After incubation for 1 hr at 37° C. in terminal deoxynucleotidyl transferase (TdT) reaction mixture, signals were visualized under a fluorescence microscope (excitation/emission wavelengths: 450-500 nm/515-565 nm). Samples were further blotted with alkaline phosphatase-conjugated anti-fluorescein antibody. Following color reaction, samples were analyzed under a light microscope.

### EXAMPLE 8

### Statistical Analysis

Data were analyzed by one-way ANOVA, followed by post hoc tests of Newman-Keula multiple comparison to determine whether there were significant differences between individual groups. Statistical significance was established when p<0.05.

The invention may be embodied in other specific forms without departing from its essential characteristics. The described embodiments are to be considered in all respects only as illustrative and not as restrictive. Indeed, those skilled in the art can readily envision and produce further embodiments, based on the teachings herein, without undue experimentation. The scope of the invention is, therefore, indicated by the appended claims rather than by the foregoing description. All changes that come within the meaning and range of the equivalence of the claims are to be embraced within their scope.

### REFERENCES

Master, C. L., Multhaup, G., Simmis, G., Pottgiesser, J., Martins, R. N. and Beyreuther, K. (1985) Embo. J. 4; 2757-63.

Selkoe, D. J. (1990) Science 248; 1058-60.

Yankner, B. A, Duffy, L. K. and Kirschner, D. A. (1990) Science 250; 279-82.

Pike, C. J., Walencewica, A. J., Glabe, C. G. and Cotman, C. W. (1991) Eur. J. Pharmacol. 207; 367-8.

Meda, L., Cassatella, M. A., Szendrei, G. I., Orvos, L., Jr., Baron, P., Villalba, M., Ferrari, D. and Rossi, F. (1995) Nature 374; 647-50.

Combs, C. K,. Karlo, J. C., Kao, S.C. and Landreth, G. E. (2001) J. Neurosci. 21; 1179-88.

Colton, C. A. and Gilbert, D. L. (1987) FEBS Lett 223; 284-8.

Klegeris, A., and McGreer, P. L. (1994) J. Neuroimmunol. 53; 83-90.

Chao, C. C., Hu, S., Molitor, T. W., Shaskan, E. G. and Peterson, P. K. (1992) J. Immunol. 149; 2736-41.

Boje, K. M. and Arora, P. K. (1992) Brain Res. 587; 250-6.

Goodwin, J. L., Uemura, E. and Cunnick, J. E. (1995) Brain Res. 692; 207-14.

Ii, M., Sunamoto, M., Ohnishi, K. and Ichimori, Y. (1996) Brain Res. 720; 93-100.

Wood, P. L. (1995) Neurol. Res. 17; 242-8.

Tanaka, M., Sotomatsu, A., Yoshida, T., Hirai, S. and Nishida, A. (1994) J. Neurochem. 63; 266-70.

Yeh, M. W., Kaul, M., Zheng, J., Nottet, H. S., Thylin, M., Gendelman,, H. E. and Lipton, S. A. (2000) J. Immunol. 164; 4265-70.

Giulian, D., Haverkamp, L. J., Li, J., Karshin, W. L., Yu, J., Tom, D., Li, X. and Kirkpatrick J. B. (1995) Neurochem. Int. 27; 119-37.

Flavin, M. P., Zhao, G. and Ho, L. T. (2000) Glia. 29; 347-54.

Minghetti, L and Levi, G. (1998) Prog. Neuobiol. 54; 99-125.

Ignarro, L. J., Cirino, G., Casini, A. and Napoli, C. (1999) J. Cardiovasc. Phamacol. 34; 879-86.

Troy, C. M., Rabacchi, S. A., Friedman, W. J., Frappier, T. F., Brown, K. and Shelanski, M. L. (2000) J. Neurosci. 20; 1386-92.

Wirtz Brugger, F. and Giovanai, A. (2000) Neuroscience 99; 737-50.

Possel, H., Noack. H., Augustin, W., Keilhoff G. and Wolf, G. (1997) FEBS Lett. 416; 175-8.

Crow, J: P. (1997) Nitric Oxide 1; 145-57.

Wang, H. and Joseph, J. A. (1999) Free Radic. Biol. Med. 27; 612-6.

Ischiropoulos, H., Zhu, L. and Beckman, J. S. (1992) Arch. Biochem. Biophys. 298; 446-51.

Maria, S. S., Lea, J. and Groves, J. T. (1997) Proc. Nail. Acad. Sci. USA 94; 14243-8.

Estevcz, A. G., Spear, N., Manuel. S. M. Barbeito, L., Radi, R. and Beckman, J. S. (1998) Prog Brain Am. 118; 269-80.

Van Dyke, K. (1997) Med. Hypotheses 48; 375-80.

Hogg, N., Darley-Usmar, V. M., Wilson, M. T. and Moncada, S. (1992) Biochem. J. 281; 419-24.

Estevez, A. G., Spear, N, Manuel, S. M., Radi, R., Henderson. C. E., Barbeito, L. and Beckman, J. S. (1998) J. Neurosci. 18; 923-31.

Mattson, M. P. (1997) Physiological Reviews 77; 1081-132.

Behl C., Davis, J. B., Lesley, R. and Schubert, D. (1994) Cell 77; 817-27.

Behl, C. (1997) Cell and Tissue Research 290; 471-80.

Keller, J. N, Kindy, M. S., Holtsberg, F. W., St Clair, D. K., Yen, H. C., Germeyer, A., Steiner, S. M., Bruce-Keller, A. J., Hutchins, J. B. and Mattson, M. P. (1998) J. Newwcat 18; 687-97.

Longo, V. D., Viola, K. L., Klein, W. L and Finch, C. E. (2000) J. Neurochem. 75; 1977-85.

McDonald, D. R., Brunden, K. R. and Landreth, G. E. (1997) J. Neurosci. 17; 2284-94.

Klegeris, A. and McGeer, P. L. (1997) J. Neurosci. Res. 49; 229-35.

Colton, C. A., Chemyshev, O. N., Gilbert, D. L. and Vitek, M. P. (2000) Ann. N.Y. Acad. Sci. 899; 292-307.

Bianca, V. D., Dusi, S., Bianchini, E., Dal Pra, L and Rossi, F. (1999) J. Biol. Chem. 274;15493-9.

Sbimohama, S., Tanino, H., Kawakami, N., Okamura, N., Kodama, H., Yamaguchi, T., Hayakawa, T., Nunomura, A., Chiba, S., Perry, G., Smith, M. A. and Fujimoto, S. (2000) Biochem. Biophys. Res. Commun. 273; 5-9.

Ii, M., Sunamoto, M., Ohnishi, K. and Ichimori, Y. (1996) Brain Res. 720; 93-100.

Salvemini, D., Wang, Z. Q., Stem, M. K., Currie, M. G. and Misko, T. P. (1998) Proc. Natl. Acad. Sci. USA 95; 2659-63.

Kim, Y. M., Chung, H. T., Kim, S. S., Han, J. A., Yoo. Y. M., Kim, K. M., Lee, G. H., Yun, H. Y., Green, A., Li, J., Simmons, R L. and Billiar, T. R. (1999) J. Neurosci. 19; 6740-7.

Chao, C. C., Molitor, T. W. and Hu, S. (1993) J. Immunol. 151; 1473-81.

Barger, S. W:, Horster, D., Furukawa, K., Goodman, Y., Krieglstein, J. and Mattson, M. P. (1995) Proc. Natl. Acad. Sci. USA 92; 9328-32.

Smith, M. A., Atwood, C. S., Joseph, J. A., and Perry, G. (2002) J. Neurosc. Res. 69:285. Marwick, C. (Sept. 27, 2000) JAMA 284(12):1503-5.

We claim:

1. A method of treating a medical condition in a subject, wherein said condition is affected by the presence of peroxynitrite, said method comprising administering to the subject a compound that decomposes peroxynitrite but does not negatively affect normal activity of microglia and other brain cells, wherein the decomposition of peroxynitrite alone is sufficient to alleviate the pathology of said condition.

2. The method of claim 1, wherein said medical condition is Alzheimer's disease.

3. The method of claim 2, wherein said treatment further comprises administering a vaccine that increases the microglial of clearing AB.

4. The method of claim 4, wherein said normal activity of microglia comprises generation of nitric oxide.

5. The method of claim 4, wherein said normal activity of microglia comprises clearance of Aβ.

6. A method of treating a medical condition in a subject, wherein said condition is affected by activated microglia, wherein said activation results in the production of peroxynitrite, said method comprising administering to the subject a compound that decomposes peroxynitrite but does not negatively affect the normal activity of microglia and other brain cells, wherein the decomposition of peroxynitrite alone is sufficient to alleviate the pathology of said condition.

7. The method of claim 6, wherein said normal activity of microglia comprises generation of nitric oxide.

8. The method of claim 6, wherein said normal activity of microglia comprises clearance of Aβ.

9. The method of claim 6, wherein said condition is Alzheimer's disease.

10. The method of claim 6, wherein said treatment further comprises administering a vaccine that increases the activity of said microglia.

11. The method of claim 6, wherein said microglia are activated by Aβ, anti-Aβ antibodies, a combination of Aβ and anti-Aβ-, or LPS.

12. A method of screening an effective test compound that decreases neuron death caused by peroxynitrite, said method comprising:
(a) providing a co-culture of microglia and neurons;
(b) exposing said co-culture to said test compound to form a test mixture;
(c) subjecting said test mixture to conditions that activate said microglia;
(d) examining said test mixture at a selected time after said subjection for the extent of neuron cell death; and
(e) measuring the extent of neuron death, wherein said effective test compound is identified as a compound that decreases neuron death relative to a control sample and does not affect normal activity of microglia.

13. The method of claim 12, wherein said conditions that activate said microglia comprise treating the co-culture with Aβ₁₋₄₂.

14. The method of claim 12, wherein said conditions that activate said microglia comprise treating the co-culture with LPS.

15. A method of identifying a mediator of LPS-activated microglia neurotoxicity, comprising:
(a) examining the effect of an inhibitor of peroxynitrite or a decomposition catalyst of peroxynitrite on a co-culture of microglia and neurons that has been treated with LPS.

16. A method of identifying a mediator of AP-activated microglia neurotoxicity, comprising:
(a) examining the effect of an inhibitor of peroxynitrite or a decomposition catalyst of peroxynitrite on a co-culture of microglia and neurons that has been treated with Aβ₁₋₄₂.

17. A method of treating a medical condition in a subject, wherein said condition is affected by the presence of TNF-α, said method comprising administering to the subject a compound that inhibits secretion of TNF-α from microglia but does not negatively affect normal activity of said microglia and other brain cells.

18. A method of treating Alzheimer's disease in a subject, said method comprising administering to the subject a compound that decomposes peroxynitrite but does not negatively affect normal activity of microglia and the normal activity of other brain cells, wherein the decomposition of peroxynitrite alone is sufficient to alleviate the pathology of said condition.

19. A method of treating Alzheimer's disease in a subject, said method comprising administering to the subject a compound that inhibits secretion of TNF-α from microglia but does not affect normal activity of said microglia and the normal activity of other brain cells, wherein the inhibition of TNF-α secretion alone is sufficient to alleviate the pathology of said condition.

20. A method of treating a medical condition in a subject, wherein said condition is affected by the presence of peroxynitrite, said method comprising administering to a subject a vaccine that increases the microglial activity of clearing of Aβ, wherein the method further comprises administering a compound that decomposes peroxynitrite but does not negatively affect the normal activity of microglia and other brain cells, wherein the decomposition of peroxynitrite alone is sufficient to alleviate the pathology of said condition.

21. The method of claim 20, wherein said compound is administered prior to, concurrently with, or after administration of said vaccine.

22. The method of claim 20, wherein said condition is Alzheimer's disease.
